(19) United States
(12) Patent Application Publication
(10) Pub. No.: US 2014/0087364 A1
(43) Pub. Date: Mar. 27, 2014

(54) METHOD AND KIT FOR DETERMINING THE TIME OF SEROCONVERSION OF A PATIENT INFECTED WITH A VIRUS

(71) Applicant: Tamar Jehuda-Cohen, Moshav Gimzo (IL)

(72) Inventor: Tamar Jehuda-Cohen, Moshav Gimzo (IL)

(21) Appl. No.: 14/006,687

(22) PCT Filed: Mar. 22, 2012
(86) PCT No.: PCT/IL12/50103

§ 371 Date: Dec. 5, 2013

Related U.S. Application Data
(60) Provisional application No. 61/466,890, filed on Mar. 23, 2011.

(51) Int. Cl. G01N 33/569

(52) U.S. Cl. CPC G01N 33/569; G01N 33/56983
USPC 435/5; 435/7.1; 435/7.92

(57) ABSTRACT

The present invention provides a method for determining the time of infection, and a method for determining if a microbial infection is in the early stages comprising the step of determining the ratio of in vitro stimulated anti-microbial immunoreactivity and un-stimulated anti-microbial immunoreactivity in blood samples from said subject and related kits.

### FIELD OF THE INVENTION

The present invention provides a method for determining the time of infection, and a method for determining if a microbial infection is in the early stages comprising the step of determining the ratio of in vitro stimulated anti-microbial immunoreactivity and un-stimulated anti-microbial immunoreactivity in blood samples from said subject and related kits.

### BACKGROUND OF THE INVENTION

There is a need for assays that can identify individuals who are at the early stages of infection with the human immunodeficiency virus (HIV). Such assays are useful inter alia for determining the stage of infection for treatment purposes.

Tests for Recent Infection (TRIs) classify infections as recently or non-recently acquired, based on the results of laboratory tests that quantify biomarkers which evolve with time after infection, sometimes supplemented by clinical information. However, tests for recent HIV infection have traditionally been based on antibody avidity, proportion or titer, for which high false recent rates (ε) or low recency durations (ω) have hindered incidence estimation. Therefore, a new and better way of identifying recent infections is needed in the art. Also, and most importantly, none of these assays have been designed for diagnostic purposes, and they are all merely statistical tools for estimating incidence.

The classification of infections by a TRI is usually based on measured biomarkers. One challenge is that evolution of these biomarkers within infected individuals exhibit inter-subject variability. In some cases, the state of recent infection is too transient for the population proportion to be estimated with good statistical power in studies with feasible samples sizes. In addition, there are often many individuals who remain classified as recently infected indefinitely or for very long periods, or who revert to a recent classification during end stage disease or under the influence of anti-retroviral treatment.

TRIs thus far proposed (such as detuned ELISAs, the BED assay and avidity assays) all crucially rely on measurements of antibody titer, avidity or HIV-specific proportion. However, these TRIs appear to be plagued by an unsatisfactory trade-off between the transient state of recent infection and false recent infections. In summary, for a TRI to be useful, the Mean Recency Duration (mean duration in the TRI-defined state of recent infection) should be large, while the False Recent Rate (the proportion of long-infected individuals who remain in the TRI-defined state of recent infection) should be small. In the case of a diagnostic tool for determining the time of infection of an individual, the ability of the assay to determine the duration of infection across populations, gender, and other variables needs to be addressed and personal variations/factors taken into account for individual diagnosis. This invention addresses the need for Infection Time Tests (ITT) for determining the time of infection and/or the duration of infection.

### SUMMARY OF THE INVENTION

In one embodiment, the present invention provides a method of determining the time of an initial microbial infection of a subject comprising the steps of: (a) determining the anti-microbial immunoreactivity in a first aliquot of a blood sample obtained from a subject, wherein a detectable anti-microbial immunoreactivity indicates that said subject is infected; (b) stimulating a second aliquot of said blood sample to produce anti-microbial antibodies in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; and (d) determining the time of said initial microbial infection based on the SI value obtained in step (c), wherein there is an inverse correlation between the SI value and the time of said microbial infection.

Thus, in one embodiment, the present invention provides a method of determining the time of an initial microbial infection of a subject comprising the steps of: (a) determining the anti-microbial immunoreactivity in a first aliquot of a first blood sample obtained from a subject, wherein a detectable anti-microbial immunoreactivity indicates that said subject is infected; (b) stimulating a second aliquot of said blood sample to produce anti-microbial antibodies in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; (d) repeating steps (a)-(c) with a second blood sample obtained from said subject at a later time; and (e) calculating the slope (or Δ) of the change in SI calculated for the first and second blood samples, wherein there is an inverse correlation between the value of said slope (or Δ) and the time elapsed since said microbial infection.

In another embodiment, the present invention provides a method of distinguishing between early versus established microbial infection in a subject comprising the steps of: (a) determining the anti-microbial immunoreactivity in a first aliquot of a blood sample obtained from a subject, wherein detectable anti-microbial immunoreactivity indicates that said subject is infected; (b) stimulating a second aliquot of said blood sample to produce anti-microbial antibodies in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; and (d) determining if the SI value obtained in step (c) is above a pre-determined threshold value for each subject, wherein a value below said threshold indicates that the subject is not in the early stages of infection and a value above said threshold indicates that the subject is in the early stages of infection, thereby distinguishing between an early and an established microbial infection in a subject.

In another embodiment, the present invention provides a method of distinguishing between early versus established microbial infection in a subject comprising the steps of: (a) determining the anti-microbial immunoreactivity in a first aliquot of a blood sample obtained from a subject, wherein detectable anti-microbial immunoreactivity indicates that said subject is infected; (b) stimulating a second aliquot of said blood sample to produce anti-microbial antibodies in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; and (d) repeating steps (a)-(c) with a second blood sample obtained from said subject at a later time; and (e) calculating the slope (or Δ) of the change in SI calculated for the first and second blood samples, wherein a value below a pre-determined threshold value indicates that the subject is not in the early stages of infection and a value above said threshold indicates that the subject is in the early stages of infection, thereby distinguishing between an early and an established microbial infection in a subject.

In another embodiment, the present invention provides a kit for determining the time of an initial microbial infection of a subject comprising: two containers for collecting whole blood samples, wherein the second container comprises a media comprising one or more activators of microbial-specific lymphocytes, non-specific lymphocytes, or a combination thereof, an assay for the detection of one or more products of said lymphocytes, and instructions for use.

In another embodiment, the present invention provides a kit for distinguishing between an early and an established microbial infection in a subject comprising: two containers for collecting whole blood samples, wherein the second container comprises a media comprising one or more activators of microbial-specific lymphocytes, non-specific lymphocytes, or a combination thereof, an assay for the detection of one or more products of said lymphocytes, and instructions for use.

### BRIEF DESCRIPTION OF THE DRAWINGS

FIG. 1: Stimulated and Unstimulated Antibody Levels. Blood specimens incubated stimulation media (Stimulated) have higher antibody levels compared to control blood specimens (Unstimulated) after seroconversion. The increased antibody levels in the stimulated sample fade with time after seroconversion.

FIG. 2: Stimulation Index (SI) Change Over Time Following Seroconversion. Simplified model for calculating time of infection and/or determining if an infection is in the early stages using the Stimulation Index (SI). Data is collected in a prospective study of high risk individuals, providing the changes to SI over time from the start of a new infection. V1=the first seropositive visit after an acute infection sample. i.e. the time of infection is “known”. The time interval between visits is equal (e.g. −1 month). “Series”=sequential blood samples from the same patient. Data from a first (A) and second (B) population are presented.

FIG. 3: Stimulation Index (SI) in Early Infections of Unknown Seroconversion Data. The first visit (V?1) takes place an unknown time after seroconversion. When, for example, V3 represents an unknown time of serocoversion, the fact that visit 3 represents early infection can be determined by the SI levels and/or the slope of SI change over time. The SI change over time in patients with unknown time of seroconversion is super-imposed (by “best fit” program) on the graph describing the SI change over time in patients with known time of seroconversion and time of infection (aka seroconversion) is thereby estimated. The time interval between visits is equal (e.g. −1 month). “Series”=sequential blood samples from the same patient.

FIG. 4: Determining the Time of Infection Using ΔSI. The time of infection is determined by “super imposing” the slope of the SI in the unknown samples over the established slopes of a set of known times of seroconversion.

### DETAILED DESCRIPTION OF THE PRESENT INVENTION

In one embodiment, the present invention provides a method of determining the time elapsed since an initial microbial infection of a subject comprising the steps of: (a) determining the anti-microbial immunoreactivity in a first aliquot of a blood sample obtained from a subject, wherein a detectable anti-microbial immunoreactivity indicates that said subject is infected; (b) stimulating a second aliquot of said blood sample to produce anti-microbial immunoreactivity in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; and (d) determining the time elapsed since said initial microbial infection based on the SI value obtained in step (c), wherein there is an inverse correlation between the SI value and the time elapsed since said microbial infection. In one embodiment, the anti-microbial immunoreactivity is anti-microbial antibody levels, cytokine levels, lymphokine levels, or a combination thereof. In one embodiment, the stimulation step is performed only if said subject is seropositive. In another embodiment, the unstimulated and stimulated blood aliquots may be processed at the same time. In another embodiment, the unstimulated and stimulated blood aliquots may be tested for antibody levels at the same time.

In one embodiment, the present invention provides a method of determining the time of a microbial infection of a subject comprising the steps of: (a) determining the anti-microbial immunoreactivity in a first aliquot of a blood sample obtained from a subject, wherein a detectable anti-microbial immunoreactivity indicates that said subject is infected; (b) stimulating a second aliquot of said blood sample to produce anti-microbial immunoreactivity in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; and (d) determining the time of said microbial infection based on the SI value obtained in step (c), wherein there is an inverse correlation between the SI value and the time of said microbial infection.

In one embodiment, the anti-microbial immunoreactivity is anti-microbial cytokine levels, lymphokine levels, antibody levels, or a combination thereof.

In one embodiment, detectable anti-microbial immunoreactivity indicates that said subject is seropositive. In another embodiment, detectable anti-microbial immunoreactivity indicates that said subject is infected.

In one embodiment, the present invention provides a method of determining the time elapsed since an initial pathogenic infection of a subject. In another embodiment, the present invention provides a method of determining the time of an initial pathogenic infection of a subject.

In one embodiment, the present invention provides a method of determining the time elapsed since an initial viral infection of a subject comprising the steps of: (a) determining the anti-viral antibody level in a first aliquot of a blood sample obtained from a subject, wherein a detectable anti-viral antibody level indicates that said subject is seropositive; (b) stimulating a second aliquot of said blood sample to produce anti-viral antibodies in vitro and determining the anti-viral antibody level in said second aliquot of said blood sample; (c) dividing a value representing the stimulated anti-viral antibody level obtained in step (b) by a value representing the anti-viral antibody level obtained in step (a), thereby determining a stimulation index (SI) value; and (d) determining the time elapsed since said initial viral infection based on the SI value obtained in step (c), wherein there is an inverse correlation between the SI value and the time elapsed since said viral infection.

In one embodiment, the present invention provides a method of determining the time elapsed since an initial retroviral infection of a subject comprising the steps of: (a) determining the anti-retroviral antibody level in a first aliquot of a blood sample obtained from a subject, wherein a detectable anti-retroviral antibody level indicates that said subject is seropositive; (b) stimulating a second aliquot of said blood sample to produce anti-retroviral antibodies in vitro and determining the anti-retroviral antibody level in said second aliquot of said blood sample; (c) dividing a value representing the stimulated anti-retroviral antibody level obtained in step (b) by a value representing the anti-retroviral antibody level obtained in step (a), thereby determining a stimulation index (SI) value; and (d) determining the time elapsed since said initial retroviral infection based on the SI value obtained in step (c), wherein there is an inverse correlation between the SI value and the time elapsed since said retroviral infection.

In one embodiment, the present invention provides a method of determining the time elapsed since an initial Human Immunodeficiency Virus (HIV) infection of a subject comprising the steps of: (a) determining the anti-HIV antibody level in a first aliquot of a blood sample obtained from a subject, wherein a detectable anti-HIV antibody level indicates that said subject is seropositive; (b) stimulating a second aliquot of said blood sample to produce anti-HIV antibodies in vitro and determining the anti-HIV antibody level in said second aliquot of said blood sample; (c) dividing a value representing the stimulated anti-HIV antibody level obtained in step (b) by a value representing the anti-HIV antibody level obtained in step (a), thereby determining a stimulation index (SI) value; and (d) determining the time elapsed since said initial HIV infection based on the SI value obtained in step (c), wherein there is an inverse correlation between the SI value and the time elapsed since said HIV infection.

In another embodiment, a method of the present invention further comprises the step of formulating a correlation between SI values and time of infection prior to step (a) based on a population of subjects for whom both SI value and time of infection is known.

In one embodiment, the SI value is analyzed in relation to another measured factor which in one embodiment, is total IgG levels, total IgM levels, total microbial specific antibody levels, total IL-2 levels, total IL10 levels, or a combination thereof in the unstimulated samples.

In another embodiment, a method of the present invention further comprises the steps of (a) determining the ratio of in vitro stimulated anti-microbial immunoreactivity and un-stimulated anti-microbial immunoreactivity in a second blood sample from said subject, wherein said ratio is the stimulation index (SI), and (b) calculating the slope (or Δ) of the change in SI calculated for the first and second blood samples, wherein the slope (or Δ) enables/assists in determining the time elapsed since said microbial infection.

Thus, in one embodiment, the present invention provides a method of determining the time of an initial microbial infection of a subject comprising the steps of: (a) determining the anti-microbial immunoreactivity in a first aliquot of a first blood sample obtained from a subject, wherein a detectable anti-microbial immunoreactivity indicates that said subject is infected; (b) stimulating a second aliquot of said blood sample to produce anti-microbial antibodies in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; (d) repeating steps (a)-(c) with a second blood sample obtained from said subject at a later time; and (e) calculating the slope (or Δ) of the change in SI calculated for the first and second blood samples, wherein there is an inverse correlation between the value of said slope (or Δ) and the time elapsed since said microbial infection.

In another embodiment, the present invention provides a method of distinguishing between early versus established microbial infection in a subject comprising the steps of: (a) determining the anti-microbial immunoreactivity in a first aliquot of a blood sample obtained from a subject, wherein detectable anti-microbial immunoreactivity indicates that said subject is infected; (b) stimulating a second aliquot of said blood sample to produce anti-microbial antibodies in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; and (d) repeating steps (a)-(c) with a second blood sample obtained from said subject at a later time; and (e) calculating the slope (or Δ) of the change in SI calculated for the first and second blood samples, wherein a value below a pre-determined threshold value indicates that the subject was not in the early stages of infection and a value above said threshold indicates that the subject was in the early stages of infection, thereby distinguishing between an early and an established microbial infection in a subject.

In another embodiment, a method of the present invention further comprises the steps of (a) determining the ratio of in vitro stimulated anti-microbial immunoreactivity and un-stimulated anti-microbial immunoreactivity in a second blood sample from said subject, wherein said ratio is the stimulation index (SI), and (b) calculating the slope (or Δ) of the change in SI calculated for the first and second blood samples, wherein there is an inverse correlation between the value of said slope (or Δ) and the time elapsed since said microbial infection.

In one embodiment, the second blood sample is taken approximately one month after the first blood sample. In another embodiment, the second blood sample is taken approximately one, two, or three weeks after the first blood sample. In another embodiment, the second blood sample is taken approximately one, two, three, four, five or six months after the first blood sample. In another embodiment, the second blood sample is taken approximately one, two, or three weeks after the first positive test and SI measurement. In another embodiment, the second blood sample is taken approximately one, two, three, four, or five months after the first positive test and SI measurement. In another embodiment, the second blood sample is taken at a time point after the first blood sample that is either potentially informative or practical in terms of patient care, as is understood in the art.

In one embodiment, specific immunoreactivity is measured in the stimulated versus the non-stimulated blood sample aliquot. In another embodiment, non-specific immunoreactivity is measured in the stimulated versus the non-stimulated blood sample aliquot. In another embodiment, both specific immunoreactivity and non-specific immunoreactivity is measured in the stimulated versus the non-stimulated blood sample aliquot. In one embodiment, the determination of the time of infection is based on the SI of the specific immunoreactivity factored by the SI of the non-specific immunoreactivity.

In another embodiment, the present invention provides a method of distinguishing between early versus established microbial infection in a subject comprising the steps of: (a) determining the anti-microbial immunoreactivity in a first aliquot of a blood sample obtained from a subject, wherein a detectable anti-microbial immunoreactivity indicates that said subject is infected; (b) stimulating a second aliquot of said blood sample to produce anti-microbial antibodies in vitro and determining the anti-microbial immunoreactivity in said second aliquot of said blood sample; (c) dividing a value representing the stimulated anti-microbial immunoreactivity obtained in step (b) by a value representing the anti-microbial immunoreactivity obtained in step (a), thereby determining a stimulation index (SI) value; and (d) determining if the SI value obtained in step (c) is above a pre-determined threshold value for each subject, wherein a value below said threshold indicates that the subject does not have an early infection and a value above said threshold indicates that the subject has an early infection, thereby distinguishing between an early and an established microbial infection in a subject.

In another embodiment, the present invention provides a method of distinguishing between early versus established viral infection in a subject comprising the steps of: (a) determining the anti-viral antibody level in a first aliquot of a blood sample obtained from a subject, wherein a detectable anti-viral antibody level indicates that said subject is seropositive; (b) stimulating a second aliquot of said blood sample to produce anti-viral antibodies in vitro and determining the anti-viral antibody level in said second aliquot of said blood sample; (c) dividing a value representing the stimulated anti-viral antibody level obtained in step (b) by a value representing the anti-viral antibody level obtained in step (a), thereby determining a stimulation index (SI) value; and (d) determining if the SI value obtained in step (c) is above a pre-determined threshold value for each subject, wherein a value below said threshold indicates that the subject does not have an early infection and a value above said threshold indicates that the subject does have an early infection, thereby distinguishing between early versus established viral infection in a subject.

In another embodiment, the present invention provides a method of distinguishing between early versus established retroviral infection in a subject comprising the steps of: (a) determining the anti-retroviral antibody level in a first aliquot of a blood sample obtained from a subject, wherein a detectable anti-retroviral antibody level indicates that said subject is seropositive; (b) stimulating a second aliquot of said blood sample to produce anti-retroviral antibodies in vitro and determining the anti-retroviral antibody level in said second aliquot of said blood sample; (c) dividing a value representing the stimulated anti-retroviral antibody level obtained in step (b) by a value representing the anti-retroviral antibody level obtained in step (a), thereby determining a stimulation index (SI) value; and (d) determining if the SI value obtained in step (c) is above a pre-determined threshold value for each subject, wherein a value below said threshold indicates that the subject was not in the early stages of infection and a value above said threshold indicates that the subject was in the early stages of infection, thereby distinguishing between early versus established retroviral infection in a subject.

In another embodiment, the present invention provides a method of distinguishing between early versus established Human Immunodeficiency Virus (HIV) infection in a subject comprising the steps of: (a) determining the anti-HIV antibody level in a first aliquot of a blood sample obtained from a subject, wherein a detectable anti-HIV antibody level indicates that said subject is seropositive; (b) stimulating a second aliquot of said blood sample to produce anti-HIV antibodies in vitro and determining the anti-HIV antibody level in said second aliquot of said blood sample; (c) dividing a value representing the stimulated anti-HIV antibody level obtained in step (b) by a value representing the anti-HIV antibody level obtained in step (a), thereby determining a stimulation index (SI) value; and (d) determining if the SI value obtained in step (c) is above a pre-determined threshold value for each subject, wherein a value below said threshold indicates that the subject was not in the early stages of infection and a value above said threshold indicates that the subject was in the early stages of infection, thereby distinguishing between early versus established HIV infection in a subject.

In one embodiment, step (d) comprises determining the mean duration of early infection which correlates to the SI value obtained in step (c) thus estimating/determining the duration of the infection and/or the time of infection.

In one embodiment, the viral infection is a retroviral infection. In one embodiment, the retrovirus is HIV. In another embodiment, it is a retrovirus. In one embodiment, the retrovirus is Alpharetrovirus, which in one embodiment is an Avian leukosis virus or a Rous sarcoma virus. In another embodiment, the retrovirus is a betaretrovirus, which in one embodiment, is a mouse mammary tumour virus. In another embodiment, the retrovirus is a gammaretrovirus, which in one embodiment, is a murine leukemia virus or feline leukemia virus. In another embodiment, the retrovirus is a deltaretrovirus which in one embodiment, is a bovine leukemia virus or the cancer-causing Human T-lymphotropic virus (HTLV), which in one embodiment, is HTLV-1, and in another embodiment, it is HTLV-2. In another embodiment, the retrovirus is a epsilonretrovirus, which in one embodiment, is a Walleye dermal sarcoma virus. In another embodiment, the retrovirus is a lentivirus, which in one embodiment, is a human immunodeficiency virus 1, Simian immunodeficiency virus, or Feline immunodeficiency virus. In another embodiment, the retrovirus is a spumavirus, which in one embodiment, is a simian foamy virus. In another embodiment, the retrovirus is a hepatitis C virus (HCV). In another embodiment, the retrovirus is a hepatitis E virus (HEV).

In one embodiment, a virus related to the methods and kits of the present invention is xenotropic murine leukemia virus (XMRV). In another embodiment, the virus is hepatitis A virus (HAV). In another embodiment, the virus is hepatitis B virus (HBV). In another embodiment, the virus is hepatitis C virus (HCV). In another embodiment, the virus is hepatitis D virus (HDV). In another embodiment, the virus is hepatitis E virus (HEV). In another embodiment, the virus is Human T-lymphotrophic virus-1 (HTLV-1). In another embodiment, the virus is any combination of the viruses disclosed hereinabove. In another embodiment, the virus is hepatitis B virus (HBV), hepatitis C virus (HCV), or hepatitis E (HEV) virus.

In another embodiment the virus is human immunodeficiency virus (HIV). In one embodiment, the HIV is HIV-1. In another embodiment, the HIV is HIV-2. In another embodiment, the HIV is HIV-0.

In one embodiment, the method further comprises the step of obtaining or collecting a blood sample from said subject prior to step (a).

In another embodiment, the present invention provides a method of determining the time elapsed since an initial microbial infection of a subject, the method comprising (a) determining the ratio of in vitro stimulated anti-microbial immunoreactivity and un-stimulated anti-microbial immunoreactivity in a blood sample from said subject, wherein said ratio is the stimulation index (SI) value and (b) determining the time elapsed since said initial microbial infection based on the SI value, wherein there is an inverse correlation between the SI value and the time elapsed since said microbial infection.

In another embodiment, the present invention provides a method of distinguishing between early versus established microbial infection in a subject, the method comprising determining the ratio of in vitro stimulated anti-microbial immunoreactivity and un-stimulated anti-microbial immunoreactivity in a blood sample from said subject, wherein if said ratio is higher than a pre-selected threshold ratio, then the subject has an early infection with said microbe.

In one embodiment, a microbe of the present invention is a pathogenic microbe.

In another embodiment, the present invention provides a method of determining the duration of a microbial infection in a subject, the method comprising (a) determining the ratio of in vitro stimulated anti-microbial immunoreactivity and un-stimulated anti-microbial immunoreactivity in a blood sample from said subject, wherein said ratio is the stimulation index (SI) value and (b) determining the duration of said microbial infection in said subject based on the SI value, wherein there is an inverse correlation between the SI value and the duration of the microbial infection.

In another embodiment, the present invention provides a method of determining the duration of a microbial seropositive state in a subject, the method comprising (a) determining the ratio of in vitro stimulated anti-microbial immunoreactivity and un-stimulated anti-microbial immunoreactivity in a blood sample from said subject, wherein said ratio is the stimulation index (SI) value and (b) determining the duration of the seropositive state of a subject based on the SI value, wherein there is an inverse correlation between the SI value and the duration of the microbial seropositive state.

In another embodiment, the present invention provides a method of estimating the time of seroconversion following a microbial seropositive antibody test in a subject, the method comprising (a) determining the ratio of in vitro stimulated anti-microbial immunoreactivity and un-stimulated anti-microbial immunoreactivity in a blood sample from said subject, wherein said ratio is the stimulation index (SI) value and (b) estimating the time of seroconversion based on the SI value, wherein there is an inverse correlation between the SI value and the time of seroconversion.

In another embodiment, the present invention provides a method of diagnosing an early state of microbial seropositivity in a subject, the method comprising determining the ratio of in vitro stimulated anti-microbial immunoreactivity and un-stimulated anti-microbial immunoreactivity in a blood sample from said subject, wherein if said ratio is higher than a pre-selected threshold ratio, then the subject is diagnosed with an early state of microbial seropositivity.

In another embodiment, the present invention provides a method of determining the recency of a microbial infection in a subject, the method comprising (a) determining the ratio of in vitro stimulated anti-microbial immunoreactivity and un-stimulated anti-microbial immunoreactivity in a blood sample from said subject, wherein said ratio is the stimulation index (SI) value and (b) determining the recency of a microbial infection in a subject based on the SI value, wherein there is an inverse correlation between the SI value and the recency of the microbial infection.

In one embodiment, the microbe is a pathogen. In one embodiment, the microbial infection is a viral infection, which in one embodiment, is a retroviral infection, which in one embodiment, is a Human Immunodeficiency Virus (HIV) infection.

In another embodiment, blood samples are taken from a subject at at least two time points, which in one embodiment, are 2 weeks apart, in another embodiment, 1 month apart in another embodiment, 2 months apart, in another embodiment, 3 months apart, in another embodiment, 4 months apart, in another embodiment, 6 months apart, in another embodiment, 1 year apart. In one embodiment, the Stimulation Index between the two or more time points is plotted and the slope of the change in Stimulation Index between the two or more time points is used to estimate the time of infection.

In another embodiment, some of the methods of the present invention comprise the steps of (a) obtaining a blood sample from said subject; (b) determining the anti-microbial immunoreactivity in a first aliquot of said blood sample, wherein a detectable anti-microbial immunoreactivity indicates that said subject is infected; (c) stimulating a second aliquot of said blood sample to produce anti-microbial antibodies in vitro and determining the anti-microbial immunoreactivity in said second aliquot of said blood sample; (d) dividing a value representing the stimulated anti-microbial immunoreactivity obtained in step (c) by a value representing the anti-microbial immunoreactivity obtained in step (b), thereby determining a stimulation index (SI) value; and (e) determining the time elapsed since said initial microbial infection based on the SI value obtained in step (d), wherein there is an inverse correlation between the SI value and the time elapsed since an microbial infection, the duration of the microbial infection, the duration of the microbial seropositive state, time of seroconversion, early infection state of a microbial infection, or a combination thereof.

In another embodiment, some of the methods of the present invention comprise the steps of (a) obtaining a blood sample from said subject; (b) determining the anti-microbial immunoreactivity in a first aliquot of said blood sample, wherein a detectable anti-microbial immunoreactivity indicates that said subject is infected; (c) stimulating a second aliquot of said blood sample to produce anti-microbial antibodies or other immune products in vitro and determining the anti-microbial immunoreactivity in said second aliquot of said blood sample; (d) dividing a value representing the stimulated anti-microbial immunoreactivity obtained in step (c) by a value representing the anti-microbial immunoreactivity obtained in step (b); and (e) determining if the quotient obtained in step (d) is above a pre-determined threshold value for each subject, wherein a value below said threshold indicates that the subject was not in the early stages of infection and a value above said threshold indicates that the subject was in the early stages of infection, thereby distinguishing between early versus established microbial infection in said subject, diagnosing an early state of microbial seropositivity, or a combination thereof.

In another embodiment, the methods of the present invention may be used to determine the early infection state of an infection, wherein the infection is a chronic infection. In one embodiment, the chronic infection is a microbial infection. In another embodiment, the chronic infection is a viral infection, which in one embodiment, is a retroviral infection. In another embodiment, the chronic infection is a bacterial infection. In one embodiment, a chronic infection is characterized by the continued presence of the infectious microbe following the initial infection. In one embodiment, the chronic viral infection is a hepatitis, herpes, infectious mononucleosis, or Cytomegalovirus (CMV) infection.

In one embodiment, the present invention provides a method of determining the early infection state of a microbial infection in a subject using the methods described herein. In one embodiment, the subject is at high risk for developing a chronic microbial infection, which in one embodiment, is HIV. In one embodiment, the subject is at risk as a result of a particular behavior, which in one embodiment, is intravenous drug use, homosexual activity, bisexual activity, sexual activity with multiple partners, receipt of blood transfusions, or a combination thereof. In another embodiment, the subject is defined high risk by living or having visited or traveled though a particular geographic location, which in one embodiment, is a continent, a region, a state, or a city. In another embodiment, the subject has a particular medical status, which in one embodiment is a hemophiliac, a subject with one or more sexually transmitted diseases, etc.

In one embodiment, the level of virus antibody in non-activated versus activated blood sample is the Stimulation Index (SI) value. In one embodiment, the SI values will be measured with varying sensitivity or amplitude depending on the detection system used. Thus, the SI values considered as “elevated” in accordance with the present invention will depend upon the precise procedure utilized. The SI values can be tested against samples obtained from individuals known to be in the early stages of infection with HIV and compared with similar samples obtained from individuals who have an established HIV infection such as, but not limited to, individuals who are known to have been infected for at least 1 year or so. Upon comparison of the results, a suitable SI value can be determined which readily distinguishes an early infection as defined herein from an established infection. Assay variation can be controlled by using the value from a standard set of sample pairs. A skilled artisan could readily use standard techniques to determine a suitable SI threshold value when using any of a variety of methods of detecting immunoreactivity to a retroviral antigen. In one embodiment, the methods of the present invention further comprise the step of determining or estimating a threshold SI value, wherein a value below said threshold indicates that the subject was not in the early stages of infection and a value above said threshold indicates that the subject was in the early stages of infection.

In one embodiment, the SI is a relative value, which in one embodiment, is measured relative to another parameter measured in the same blood sample. In one embodiment, the other parameter to which the SI value is compared is taken from the stimulated aliquot, the unstimulated aliquot, or from both the stimulated and non-stimulated aliquots. In another embodiment, the SI is an absolute value, which in one embodiment, is the change in antibody level or the change in antibody level compared to the unstimulated sample. In another embodiment, the SI is a combination of absolute and relative values. In one embodiment, the SI is a one-time value, meaning that it is obtained from a single blood sample taken at a single point in time. In another embodiment, the changes in SI over time in one or more additional (i.e. other than the first) blood samples taken at two or more separate time points are used to determine the time of infection.

In one embodiment, detecting “immunoreactivity” comprises measuring antigen-induced secretions by B cells and T cells, where in one embodiment, antibody, cytokine, lymphokine, or a combination thereof, are secreted.

In one embodiment, a sample of the present invention is obtained from a bodily fluid, such as fresh whole blood in which a single aliquot is activated and the rest of the sample is not activated, as described herein, or in another embodiment, a sample is a pair of plasma samples, in which one of the plasma pair was from activated and the other plasma pair was from not activated blood. In one embodiment, the plasma and stimulated-plasma are stored “properly”, which in one embodiment, is at a temperature of 4° C. (for short term storage of days), or in another embodiment, at a temperature of −20° C. or −80° C. (for long term storage of over a week), as is well known in the art. In one embodiment, the plasma may be stored for up to 2 days. In another embodiment, the plasma may be stored for up to 7 days. In another embodiment, the plasma may be stored for up to 14 days. In another embodiment, the plasma may be stored for up to 1 month. In another embodiment, the plasma may be stored for up to 6 months. In another embodiment, the plasma may be stored for up to 12 months. In another embodiment, the plasma may be stored for up to 6 years.

In one embodiment the HIV can be any strain or isolate. Preferably, the HIV is selected from the group consisting of HIV-1, HIV-2, or a combination thereof.

In one embodiment, the subject is a mammal, which in one embodiment, is a primate, which in one embodiment, is a human.

In one embodiment, a method of the present invention requires the determination of anti-microbial antibody levels in a tissue sample. In one embodiment, the tissue sample is a blood sample. In another embodiment, the tissue sample is obtained from the gum or cheek of the subject.

In one embodiment, a method of the present invention comprises determining the anti-microbial immunoreactivity in an aliquot of a blood sample. In one embodiment, an “aliquot” is a portion of the total amount of a blood sample. In one embodiment, the aliquots used in the methods of the present invention are of equal volume or dilution. In one embodiment, duplicate blood samples are used in the methods of the present invention. In one embodiment, the first and second aliquots of a blood sample are portions of a single blood sample drawn from a single subject at a single time point.

In another embodiment, a single aliquot of the tissue or blood sample may be used to determine both “baseline” antibody levels and stimulated antibody levels, wherein a tissue sample, such as blood is drawn into a container comprising the activator described herein and the cells are sedimented (by regular G force, or by a short centrifugations at low speed). A small aliquot of the plasma supernatant is removed for later testing of the initial levels of HIV/HCV/retrovirus/virus/pathogen/microorganism. The rest is incubated with the activator for several days. The levels of the antibodies against HIV/HCV/retrovirus/virus/pathogen/microorganism for the aliquot removed at Time0 is measured on the same assay with an aliquot of blood or tissue removed after the incubation. The two measurements are compared. In one embodiment, the delta is calculated, in another embodiment, the ratio of signals or levels, is calculated, in another embodiment, the ratio of IgM to IgG of antibodies against HIV/HCV/retrovirus/virus/pathogen/microorganism is calculated, etc.

In one embodiment, the methods of the present invention comprise calculating the stimulation index (SI) by comparing the value representing the stimulated anti-microbial antibody level obtained from said second aliquot and the value representing the initial anti-microbial antibody level obtained from the first aliquot for each sample. In one embodiment, the SI is calculated by calculating the ratio of stimulated to non-stimulated anti-microbial antibody levels. In another embodiment, the SI is calculated by calculating the difference between stimulated and non-stimulated anti-microbial antibody levels.

In accordance with the present invention, a blood sample is drawn into a test tube, which in one embodiment, is tissue culture treated, a vacuum-tube, a bottle, a well (as part of a multi well plate or as a single well/plate) or a flask, containing an effective concentration of a solution of a activators (such as mitogens, cytokines, lymphokines, and combinations thereof as described herein). The blood sample to be tested is cultured in vitro in the presence of any combination of activators of lymphocytes to achieve the same function.

In one embodiment, the step of determining anti-microbial immunoreactivity comprises performing an antibody assay on each aliquot of said blood samples. In one embodiment, an antibody assay comprises exposing each of said blood samples to a viral antigen thereby allowing an antigen-antibody immune complex to form and detecting said antigen-antibody immune complex. In one embodiment, detection of the antigen-antibody immune complex is semi-quantitative.

In one embodiment, an antigen is a compound, composition, or substance that can stimulate the production of antibodies or a T cell response in a subject, including compositions that are injected or absorbed into the subject. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. In one embodiment, T cells respond to the epitope, when the epitope is presented in conjunction with an MHC molecule, or a stand alone, or bound peptide. Epitopes can be formed both from contiguous amino acids or noncontiguous amino acids juxtaposed by tertiary folding of a protein. An epitope typically includes at least 3, and more usually, at least 5, about 9, or about 8-10 amino acids in a unique spatial conformation.

In one embodiment, a peptide is 6-200 amino acids long. In another embodiment, a peptide is 2-50 amino acids. In another embodiment, a peptide is 15-50 amino acids. In another embodiment, a peptide is 1-100 amino acids. In another embodiment, a peptide is about 3-30 amino acids. In another embodiment, a peptide is 5-20 amino acids.

In one embodiment, said antigen is added to said culture to shorten the incubation time and/or to provide diagnosis in situ. In another embodiment, the antigen-antibody immune complex is detected on a solid phase support, carrier, or solid base, which in one embodiment, is a nitrocellulose strip, a set of labeled or colored beads, or any other carrier. In one embodiment, the carrier may comprise beads with different densities, sizes, labels, colors, fluorescence, as is known in the art.

After incubation, an aliquot is taken from the supernatant and is then assayed for the presence of desired antibodies using standard ELISA procedures and/or EIA and/or any other semi-quantitative antibody detection system, which in one embodiment is a Chemiluminescence, or chip system. In one embodiment, the assay is an enzyme immunoassay (ETA) including enzyme-linked immunosorbent assay (ELISA), radioimmunopreciptation assay (RIPA), particle agglutination assay or immunofluorescence assay (IFA).

In one embodiment, the antibody assay is an enzyme linked immunosorbent assay, a blot, a chemi-illuminesense assay, a luminescence assay, or an immunofluorescence assay, a peptide-chip-array, or an antibody chip array. In one embodiment, the antibody assay is any semi-quantitative assay for HIV antibodies known in the art, total or specific.

If the sample is to be assayed at a later date, the supernatant fluid may be collected, frozen and stored.

General Techniques:

Unless otherwise indicated, the immunological techniques utilized in the present invention are standard procedures, well known to those skilled in the art. Such techniques are described and explained throughout the literature in sources such as, Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

General Methods for Detecting an HIV Infection:

Many techniques have been developed for detecting an HIV infection. At least some of these procedures are commercially available in “kit” form. Many of the techniques are generally described in HIV: A Practical Approach (Volume 1: Virology and Immunology. Ed. Jonathan Karn, IRL Press); AIDS Testing: A comprehensive guide to technical, medical, social, legal, and management issues (Ed. Gerald Schochetman and J. Richard George. 2.sup.nd Edition. Springer-Verlag, 1994); Gallo et al. (1986) and Mylonakis et al. (2000). An overview of at least some of these techniques is provided below. Furthermore, at least some of these techniques, including those of the claimed invention, can readily be adapted to be performed using nanocrystals such as those described in WO 00/27365, U.S. Pat. No. 6,207,392, Nolan and Sklar (2002), and Han et al. (2001).

Enzyme Immunoassay (EIA) or Enzyme-Linked Immunosorbent Assay (ELISA) Methodology:

ELISA detection systems have been used routinely in EIAs for many years in the detection of HIV infection by showing the presence of anti-HIV antibodies. Furthermore, there are many licensed manufacturers of EIAs and ELISAs for detecting antibody to HIV. The sensitivity of third generation EIAs is close to 100 percent when any anti-HIV-antibody is present in peripheral blood. However, these assays cannot differentiate between the earliest stages of infection and established infection.

EIA methodology involves the following steps. HIV-antigens are purified from viral lysate, prepared by recombinant DNA technology or peptide synthesis and are coated onto the wells of microwell plates or onto other matrixes such as beads to form the “solid phase” of the assay. The serum of an individual is added to the well. Antibody, if present, reacts with the antigen, and the other well contents are then washed away. An indicator reagent consisting of an anti-human antibody bound to an enzyme or other detection system is added to the well. If the serum contained HIV-specific antibodies, these will remain attached to the solid-phase antigen, and the enzyme-conjugated anti-human antibody will attach to these antibodies and thus to the solid phase. Another washing step follows. If the individual's serum contains antibody to HIV, the enzyme remains attached through antibody to the solid phase and is available to catalyze a color-producing reaction when an appropriate substrate is added to the well. The color change is measured in a spectrophotometer. Absorbance values above a cut-off value calculated from control samples are considered reactive. Within the linear (or reactive) range of the assay, the absorbance values are directly related to the levels of antibodies in the tested sample.

This basic methodology has been adapted to encompass a wide variety of assay formats including, both antigen and antibody capture assays as well as antigen and antibody competition assays.

Immuno Transfers—Western Blots and any Other Antigen Blot:

Blots are another form of EIA which have been commonly used for establishing the presence of true anti-HIV antibodies. Several commercially produced kits are available. Certain blots may be used in a semi-quantitative way.

Particle Agglutination Assays:

Antigen or antibody labeled latex particles, sepharose, polyurethane microcapsules, colloidal gold or red blood cells have been employed to produce a wide range of immuno-agglutination assays. Particles can be obtained commercially with a large range of surface chemistries allowing for great flexibility when coupling them to either antibody or antigen. These techniques are typically used in rapid assay formats that are usually scored visually, but are also adapted to automation and semi-quantification.

### Immunofluorescence Assay (IFA)

The IFA for HIV-antibody is more technically demanding and more expensive than Western blots. Because virtually all the antigens present in an infected cell are available for reaction with the test specimen, it is a very sensitive assay. It is a procedure familiar to many laboratories because it is used for detecting antibodies to a wide variety of viral and bacterial antigens.

Basically, the technique involves the following steps. A suspension of a lymphocyte cell culture infected with HIV is placed on a microscope slide, air-dried, and fixed in acetone or methanol. Uninfected control cells are added to the suspension or put in separate spots on the slide to provide a means for detecting non-specific reactions (fixed slides can be made in large batches and stored frozen or desiccated.) Diluted test sera are added to the cell spots, the slide is washed, incubated again with fluorescein-conjugated anti-human globulin, washed again, and then inspected for fluorescein fluorescence using an ultraviolet microscope.

Typical localized fluorescence of infected cells occurs after reaction with positive sera. Little or no fluorescence occurs with negative sera. Non-specific reactions (such as those caused by antinuclear antibody) are recognized because of fluorescence in uninfected control cells.

Radioimmunoprecipitation:

The radioimmunoprecipitation assay is used primarily in research. It is generally too technically demanding for routine use in clinical laboratories. Radioimmunoprecipitation is especially sensitive for antibodies to the higher molecular weight major envelope glycoproteins gp160 and gp120, which some Western blot techniques miss. The principle of RIPA involves competitive binding of radiolabeled antigen and unlabeled antigen to a high-affinity antibody. The antigen is generally labelled with a gamma-emitting isotope such as .sup.125I. The labelled antigen is mixed with antibody at a concentration that just saturates the antigen-binding sites of the antibody molecule, and then increasing amounts of unlabeled antigen of unknown concentration are added. The antibody does not distinguish labelled from unlabeled antigen, and so the two kinds of antigen compete for available binding sites on the antibody. With increasing concentrations of unlabeled antigen, more labelled antigen will be displaced from the binding sites. By measuring the amount of labelled antigen free in solution, it is possible to determine the concentration of unlabeled antigen.

In one embodiment, the terms “antibody” and “immunoglobulin” are used interchangeably herein. These terms are well understood by those in the field, and refer to a glycosylated (comprising sugar moieties) protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The term “antibody” also includes any protein or peptide containing molecule that comprises at least a portion of an immunoglobulin molecule, such as, but not limited to, one complementarity determining region (CDR) of a heavy chain or light chain constant region, a framework region, or any portion thereof. Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions or classes, e.g., gamma (of about 330 amino acids). The subunit structures and three-dimensional configurations of different classes of immunoglobulins are well known.

In one embodiment, an “antigen” includes a full length retroviral protein, a derivative of a full-length retroviral protein, such as but not limited to, a protein fragment or a synthetic peptide that comprises an amino acid sequence corresponding to a part or parts of a full-length retroviral protein, including any modified fragment or synthetic peptide having a ligand attached thereto.

In one embodiment, the stimulating step in the methods of the present invention comprises incubating a second aliquot of the subject's blood samples in a media comprising one or more activators of virus-specific cells. In one embodiment, the stimulating step comprises inducing polyclonal activation of peripheral blood mononuclear cells. In one embodiment, the stimulating step comprises inducing HIV specific activation of peripheral blood mononuclear cells. In another embodiment, the stimulating step comprises inducing polyclonal activation of lymphocytes. In one embodiment, the virus-specific cells are B-lymphocytes. In another embodiment, the virus-specific cells are T-lymphocytes.

In one embodiment, an activated blood sample comprises both antibodies produced in vivo and antibodies produced by in vitro stimulation.

In one embodiment, an activator is a stimulant. In one embodiment, an aliquot is stimulated, while in another embodiment, it is activated.

In one embodiment, the activator of the present invention stimulates blood to produce anti-microbial antibodies, while in another embodiment, the activator stimulates blood to secrete anti-microbial antibodies.

In one embodiment, an “activator” for use in the compositions and methods of the present invention is a substance that induces the activation of B cells and/or T cells, and these cells could be just primed in vivo, or in the state of blasts, or as memory cells in the blood sample. In one embodiment, the substance is a protein, while in another embodiment, it is a peptide, a nucleic acid molecule, a glycoprotein, etc. In one embodiment, “activation” of cells comprises inducing proliferation of cells, differentiation of cells, enhancement of cellular activity (in one embodiment, antibody production), secretion of various lymphokines and/or cytokines, or a combination thereof.

In one embodiment, the activator for use in the compositions and methods of the present invention activates non-secreting cells, in one embodiment, or not fully activated cells, in another embodiment, or not fully differentiated cells, in another embodiment, or memory cells, in another embodiment.

In one embodiment, the activator is a mitogen. In one embodiment, a “mitogen” is a chemical substance, or a mixture of substances. In one embodiment, the mitogen is a biochemical substance, or a mixture of biochemical substances. In one embodiment, a mitogen is one or more proteins, glycoproteins, or a combination of several proteins and glycoproteins with or without other biochemical moieties, that encourages a cell to commence cell division, triggering mitosis. In one embodiment, a mitogen triggers signal transduction pathways in which mitogen-activated protein kinase is involved, leading to mitosis. In one embodiment, mitogens of the present invention are used to induce mitosis, activation, differentiation, or a combination thereof, in memory B cells or virus-primed B cells and/or virus-primed T-cells. In one embodiment, mitogens of the present invention are used to induce the formation of antibody secreting blast cells, or plasma cells, from primed differentiating B cells and/or memory B cells. In another embodiment, mitogens of the present invention are used to induce the formation of plasma cells from primed, yet “silenced” or “tolerized”, or suppressed, B-cells and/or memory B cells.

In one embodiment, the activator of the compositions and methods of the present invention induces the activation of non-secreting B or T cells that are specific for the virus of interest. In another embodiment, the activator of the present invention induces the expression of viral specific antibodies. In another embodiment, the activator of the present invention induces the transfer from non-secreting B cells to secreting B cells, which in one embodiment, are blasts or plasma cells.

In one embodiment, an activator used in the methods and kits of the present invention enhances blast cell division, which in one embodiment, enhances the production of antibodies and, in another embodiment, enhances differentiation of B cells into plasma cells. In another embodiment, an activator used in the methods and kits of the present invention enhances blast cell division, enhances the production of antibodies, enhances differentiation of B cells into plasma cells, or a combination thereof. In one embodiment, activated B cell blasts secrete antibody and undergo cell division. In one embodiment, plasma cells secrete antibody and do not proliferate.

In one embodiment, viral antigens are used in conjunction with activators to induce activation of non-secreting B cells. Thus, in one embodiment, the compositions of the present invention additionally comprise one or more antigens specific to the virus of interest which, in one embodiment, aids or enhances the transfer from non-secreting B and/or T cells to secreting B and/or T cells, which in one embodiment, are blasts or plasma cells. Similarly, the methods of the present invention may comprise incubating a blood sample in a media containing one or more mitogens and one or more viral antigens.

In one related aspect, the activator used in the invention provided herein can be either T-cell dependent or T-cell independent. In one embodiment, the activator used in the compositions and methods of the present invention acts on T-cells, B-cells, or both T cells and B cells. In one related aspect, the activator used to induce activation of non-secreting B cells and the expression of virus specific antibodies is a mitogen, which in one embodiment, is pokeweed mitogen, which in one embodiment, stimulates both B- and T-cells. Other mitogens can be used in practicing the present invention and include, but are not limited to, lectins, such as, concanavalin A, which in one embodiment acts on T cells; bacterial endotoxins, which in one embodiment, is lipopolysaccharide (LPS), which in one embodiment, acts on B cells. In another embodiment, the mitogen is phytohaemagglutinin (PHA), which in one embodiment, acts on T cells. In another embodiment, the mitogen is leucoagglutinin (PHA-L), while in another embodiment, the mitogen is Pisum sativum agglutinin (PSA).

In another embodiment, the activator used in the composition and methods of the present invention is a cytokine, which in one embodiment is a signaling molecule secreted by specific cells of the immune system and glial cells. In one embodiment, said cytokine is an interleukin or interferon. In one embodiment, the cytokine is a lymphokine. In one embodiment, said lymphokine is Interleukin 1, Interleukin 2, Interleukin 3, Interleukin 4, Interleukin 5, Interleukin 6, Interleukin 10, Interleukin 12, Granulocyte-macrophage colony-stimulating factor, Interferon-gamma, TNF (tumor necrosis factor), or a combination thereof.

In another embodiment, the activator used in the composition and methods of the present invention is a bacterially derived lipid A, a viral-derived peptide, a virus, a biological agent, an anti-immunoglobulin reagent, an antibody against a B and/or T-lymphocyte cellular domain, or a combination thereof. In another embodiment, the activator used in the composition and methods of the present invention is a viral-derived peptide, lectin, bacterial endotoxin, a virus, lipid A, a cytokine, or a lymphokine. In another embodiment, the activator may be a combination of the activators described herein. In one embodiment, the activator is a microbial-derived peptide, protein, glycoprotein, lipoprotein, etc. In one embodiment, the microbial-derived peptide, protein, glycoprotein, or lipoprotein is a viral-derived peptide, protein, glycoprotein, or lipoprotein.

In one related aspect, stimulation of cells is achieved by using antibodies against cellular membrane domains. In another embodiment, cells are stimulated by using antibodies against a B-lymphocyte cellular domain, which in one embodiment is a membrane B-lymphocyte cellular domain. In one embodiment, the antibody is anti-IgD, which in one embodiment, is membrane-expressed by: naïve B cells, initially primed B cells, and memory cells. In one embodiment, plasma cells do not express membrane IgD. In one embodiment, primed B cells that have not fully differentiated to plasma cells can be stimulated or activated by contacting them with anti-IgD. In another embodiment, the antibody is anti-IgM. In another embodiment, the antibody is directed against a B cell cellular domain (CD). In another embodiment, the antibody is directed against a T cell CD.

In one embodiment, the membrane B-lymphocyte cellular domain is IgG, IgA, IgE, CD19, or any other membrane structure/domain known in the art. In another embodiment, the membrane B-lymphocyte cellular domain is CD21 or CD81.

In one embodiment, the antibody for use in the methods and compositions of the present invention comprises anti-IgD, anti-IgG, anti-IgA, anti-IgE, or anti-CD19, or anti-CD10, anti-CD23, anti-CD25, and anti-CD40.

In one embodiment, the antibody class used to stimulate a non-secreting cell includes, but is not limited to, an antibody from the IgG, IgD, IgA, or IgE class.

In another aspect, stimulation of non-secreting B cells, which in one embodiment, are memory cells, to secreting B cells, which in one embodiment, are blasts or plasma cells results in the transformation of the cell to an antibody-secreting blast or plasma cell, whereby the blast or plasma cell secretes antigen-specific antibodies.

In a related aspect, the B lymphocyte of the methods provided herein is a non-secreting B-lymphocytic cell. In another related aspect, the T lymphocyte is a non-secreting T-lymphocytic cell. In yet another related aspect, the activator provided herein activates a non-fully activated B-lymphocytic cell. In another embodiment, the activator activates a non-fully activated T-lymphocytic cell, and in another embodiment the activator activates both T and B cells.

In one embodiment, the early infection determination of the present invention has a low false early rate. In one early recent rate is under 5%. In another embodiment, the false early rate is under 4%. In another embodiment, the false early rate is under 3%. In another embodiment, the false early rate is under 2%. In another embodiment, the false early rate is under 1%.

In another embodiment, the determination of early infection of the present invention has a high mean early infection duration. In one embodiment, the mean early infection duration is approximately a year. In another embodiment, the mean early infection duration is approximately 11 months. In another embodiment, the mean early infection duration is approximately 10 months. In another embodiment, the mean early infection duration is approximately 9 months. In another embodiment, the mean early infection duration is approximately 8 months. In another embodiment, the mean early infection duration is approximately 7 months. In another embodiment, the mean early infection duration is approximately 6 months. In another embodiment, the mean early infection duration is approximately 5 months. In another embodiment, the mean early infection duration is approximately 4 months. In another embodiment, the mean early infection duration is approximately 3 months. In another embodiment, the mean early infection duration is approximately 2 months. In another embodiment, the mean early infection duration is approximately 1 month. In another embodiment, the mean early infection duration is approximately 70 days. In another embodiment, the mean early infection duration is approximately 60 days. In another embodiment, the mean early infection duration is approximately 45 days. In another embodiment, the mean recency duration is approximately 30 days. In another embodiment, the mean early infection duration is approximately 14 days. Thus, in one embodiment, an infection will be classified as early for diagnostic purposes if it occurred within one of the time frames described hereinabove.

In one embodiment, the SI is calculated only for subjects who have undergone seroconversion. In one embodiment, the SI is calculated only for subjects whose unstimulated blood sample (in one embodiment, the first aliquot) gave results above the cut-off of the anti-retroviral antibody assay.

In one embodiment, the mean early infection of virus infection in said population is determined. According to this aspect and in one embodiment, the early infection state for each sample in days, weeks, months, or years is calculated based on the ratio of stimulated to unstimulated anti-retroviral antibody levels, and the mean early infection of the population is calculated as is known in the art.

In one embodiment, an SI threshold is chosen that provides a low rate of false early infection determination (i.e. high diagnostic specificity). In one embodiment, the SI threshold is 1.5. In another embodiment, the SI threshold is 1.4. In another embodiment, the SI threshold is 1.3. In another embodiment, the SI threshold is 1.2. In another embodiment, the SI threshold is 1.1. In another embodiment, the SI threshold is 1.0. In another embodiment, the SI threshold is 0.95

In one embodiment, the device using an immune-stimulation technology is a commercially available tissue culture tube with a special medium which enhances antibody production in vitro in a whole blood sample. As soon as there are, for example, HIV primed B-cells in the blood, (i.e. within days of HIV infection), it is possible to get anti-HIV antibodies produced by them in vitro, at levels detectable by the currently available kits. Current serology measures the levels of HIV-specific antibodies in the blood sample. These levels are of antibodies produced in vivo. Pre-treating the blood sample in the culture tube produces a plasma sample that contains in it, in addition to the antibodies already in the plasma, the antibodies produced in vitro, during the culture step. Antibodies against HIV can be induced in vitro (produced by HIV primed B cells) within days after infection and prior to their appearance/detection in the blood. This enables earlier detection of the infection, using the currently available assays and kits for antibody detection. Thus, using the stimulated plasma as the tested sample gives a better measure of prevalence. Clinical studies have been conducted in several countries around the world showing improved diagnostic sensitivity by using stimulated plasma.

In another embodiment, the present invention provides a kit for determining the time elapsed since an initial microbial infection of a subject comprising: two aliquot containers for collected whole blood samples, wherein the second container comprises a media comprising one or more activators of microbial-specific lymphocytes, an assay for the detection of microbial specific antibodies, and instructions for use.

In another embodiment, the present invention provides a kit for determining the time elapsed since an initial microbial infection of a subject comprising: two aliquot containers for collected whole blood samples, wherein the second container comprises a media comprising one or more activators of non-specific lymphocytes, an assay for the detection of one or more products of said lymphocytes, and instructions for use.

In another embodiment, the present invention provides a kit for determining the time elapsed since an initial microbial infection of a subject comprising: two aliquot containers for collected whole blood samples, wherein the second container comprises a media comprising one or more activators of microbial-specific or non-specific lymphocytes, an assay for the detection of one or more products of said lymphocytes, and instructions for use.

In another embodiment, the present invention provides a kit for distinguishing between early versus established microbial infection in a subject comprising: two containers for collecting whole blood samples, wherein the second container comprises a media comprising one or more activators of microbial-specific, an assay for the detection of microbial specific antibodies, and instructions for use.

In another embodiment, the present invention provides a kit for distinguishing between early versus established microbial infection in a subject comprising: two containers for collecting whole blood samples, wherein the second container comprises a media comprising one or more activators of non-specific lymphocytes, an assay for the detection of one or more products of said lymphocytes, and instructions for use.

In another embodiment, the present invention provides a kit for distinguishing between early versus established microbial infection in a subject comprising: two containers for collecting whole blood samples, wherein the second container comprises a media comprising one or more activators of microbial-specific lymphocytes, non-specific lymphocytes, or a combination thereof, an assay for the detection of one or more products of said lymphocytes, and instructions for use.

In another embodiment, the present invention provides a kit for determining the time elapsed since an initial viral infection of a subject comprising: two containers for collecting whole blood samples, wherein the second container comprises a media comprising one or more activators of viral-specific or non-specific lymphocytes, an assay for the detection of viral specific antibodies, and instructions for use.

In another embodiment, the present invention provides a kit for distinguishing between early versus established viral infection in a subject comprising: two containers for collecting whole blood samples, wherein the second container comprises a media comprising one or more activators of viral-specific or non-specific lymphocytes, an assay for the detection of viral specific antibodies, and instructions for use.

In another embodiment, the present invention provides a kit for determining the time elapsed since an initial retroviral infection of a subject comprising: two containers for collecting whole blood samples, wherein the second container comprises a media comprising one or more activators of retroviral-specific or non-specific lymphocytes, an assay for the detection of retroviral specific antibodies, and instructions for use.

In another embodiment, the present invention provides a kit for distinguishing between early versus established retroviral infection in a subject comprising: two containers for collecting whole blood samples, wherein the second container comprises a media comprising one or more activators of retroviral-specific or non-specific lymphocytes, an assay for the detection of retroviral specific antibodies, and instructions for use.

In another embodiment, the present invention provides a kit for determining the time elapsed since an initial Human Immunodeficiency Virus (HIV) infection of a subject comprising: two containers for collecting whole blood samples, wherein the second container comprises a media comprising one or more activators of HIV-specific or non-specific lymphocytes, an assay for the detection of HIV specific antibodies, and instructions for use.

In another embodiment, the present invention provides a kit for distinguishing between early versus established Human Immunodeficiency Virus (HIV) infection in a subject comprising: two containers for collecting whole blood samples, wherein the second container comprises a media comprising one or more activators of HIV-specific or non-specific lymphocytes, an assay for the detection of HIV specific antibodies, and instructions for use.

In one embodiment, kits of the present invention may comprise a packaged combination of reagents in predetermined amounts with instructions for performing a method of the invention. In one embodiment, the kit may comprise suitable reagents for detecting a labeled microbial antigen. For example, when the label is an enzyme, the kit will include substrates and cofactors required by the enzyme (e.g., a substrate precursor which provides the detectable chromophore or fluorophore). In addition, other additives may be included such as stabilizers, buffers and the like. The relative amounts of the various reagents may be varied widely to provide for concentrations in solution of the reagents which substantially optimize the sensitivity of the assay. The reagents may be provided as dry powders, usually lyophilized, including excipients which on dissolution will provide a reagent solution having the appropriate concentration.

Microbial antigen for use in kits of the present invention can be provided/obtained from any source known in the art. For example, microbial antigen can be produced using recombinant methods such as those known in the art. Alternatively, microbial antigen can be purchased from a commercial supplier.

In one embodiment, the container of the kit of the present invention is for retaining tissue samples, or in another embodiment, holding, processing, storing, maintaining or collecting tissue samples.

Kits are also provided that are useful as a positive control for the diagnostic assays. For isolation and purification of anti-viral antibodies, the kit can contain viral proteins/antigens coupled to beads (e.g., sepharose beads or nanobeads or other nano-structures). Kits can be provided which contain the antibodies for detection and quantitation of anti-viral antibodies in vitro, e.g. in an ELISA, peptide microarray, lab-chip, bio-chip, nano-based chip or a Western blot. As with the article of manufacture, the kit comprises a container and a label or package insert on or associated with the container. The container holds a composition comprising at least one antigen recognized by the anti-viral antibodies. Additional containers may be included that contain, e.g., diluents and buffers, control antibodies. The label or package insert may provide a description of the composition as well as instructions for the intended in vitro or diagnostic use.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

In one embodiment, a viral infection can be detected using a diagnostic kit. In one embodiment, the diagnostic kit is one currently available. In one embodiment, a kit of the present invention may be used in conjunction with a diagnostic kit for a particular viral infection. In another embodiment, a kit of the present invention may be used in conjunction with a currently available diagnostic kit for a viral infection.

The method of the present invention includes optionally separating the blood cells from the fluid portion of the blood so that the presence of antibodies, or the presence of antibody-producing cells can be determined. The separation of the blood cells from the fluid portion of the blood can be done by any of several methods well known to those of ordinary skill in the art, including centrifugation or density dependent sedimentation. In one embodiment, the blood cells are not physically separated from the fluid. In another embodiment, peripheral blood mononuclear cells (PBMCs), B-lymphocytes and T-lymphocytes may be separated from the blood prior to culture and assay. Methods of B cell and T cell enrichment are well known in the art and can be carried out by methods that include, but are not limited to, density dependent sedimentation, and/or cell sorting/FACS. After incubation of the tissue with the mitogen, fluid from the top of the blood can easily be extracted and tested for antibody. Optionally, the red blood cells can be lysed either by mild osmotic shock or with a mild detergent. In this way, the white blood cells remain viable. Another method would be to sediment the white blood cells via density, or density gradient.

Generally, the results of a test or assay according to the invention can be presented in any of a variety of formats. The results can be presented in a qualitative fashion. For example, the test report may indicate only whether or not a particular virus-specific antibodies were detected, perhaps also with an indication of the limits of detection. The results may be presented in a semi-quantitative fashion. For example, various ranges may be defined, and the ranges may be assigned a score (e.g., 1+ to 4+) that provides a certain degree of quantitative information. Such a score may reflect various factors, e.g., the number of virus detected, the intensity of the signal (which may indicate the level of expression of virus specific B cells, or T cells), etc. The results may be presented in a quantitative fashion, e.g., as a percentage of cells in which the virus specific antibodies are detected, as a viral specific antibody concentration (as determined via different antibody binding/detection assay), etc. As will be appreciated by one of ordinary skill in the art, the type of output provided by a test will vary depending upon the technical limitations of the test and the biological significance associated with detection.

In one embodiment of the present invention, whole blood is collected in a blood collection tube containing culture medium and mitogen. The blood samples are then incubated with an approximately 1:50-1:500 final dilution of pokeweed mitogen at a concentration of 0.1-2×10⁶ viable cells per ml for four days at 37.degree. C. in a 3-10% CO₂ humidified atmosphere. The blood is then centrifuged and the supernatant fluid is collected and assayed within approximately 24 hours for reactive antibodies by ELISA, lateral flow, and/or blot techniques. In the alternative, an aliquot of fluid may be taken directly from the sample. Each sample should be screened for antibody by lateral flow (Rapid test) or ELISA first, samples considered positive may then be subjected to an additional test, e.g. blot analysis.

In one embodiment, the methods of the present invention comprise the steps described. In another embodiment, the methods of the present invention consist essentially of the steps described. In another embodiment, the methods of the present invention consist of the steps described. In one embodiment, the compositions of the present invention, which in one embodiment, are kits comprise the elements described. In another embodiment, the compositions of the present invention, which in one embodiment, are kits consist essentially of the elements described. In another embodiment, the compositions of the present invention, which in one embodiment, are kits consist of the elements described.

In one embodiment, the methods and kits of the present invention may be used in conjunction with other methods of determining the recency of a microbial infection known in the art.

### Example 1

### Test for Recent HIV Infection Using Stimulation Devices

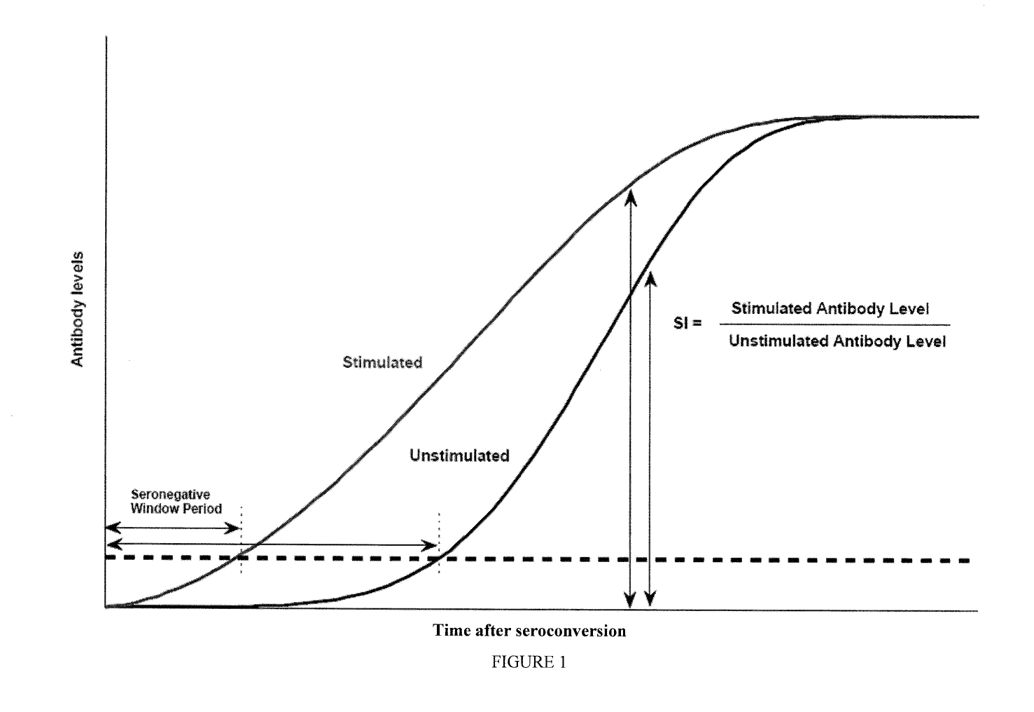

An HIV infection that is in its Seronegative Window Period, namely the period between acquiring the infection and the time of serocoversion at which antibody levels have reached measurable levels, is undetectable by diagnostic tests such as enzyme linked immunosorbent assay (ELISA)/enzyme immunoassay (EIA). To mitigate the effect of this Seronegative Window Period in producing false negative results, stimulation methods and/or stimulation devices were developed to enhance antibody detection when using existing HIV diagnostic tests. The breakthrough stimulation methods and/or stimulation devices stimulates in vivo primed specific immune cells to produce antibodies in vitro, resulting in antibody levels reaching detectable levels sooner after infection, and hence reducing the Seronegative Window Period, as illustrated in FIG. 1.

An unexpected feature of the stimulation methods and/or stimulation devices is that the increased antibody levels in a blood specimen incubated in stimulation methods and/or stimulation devices compared to control blood specimens fades with time after seroconversion (FIG. 1). Comparison of the antibody levels in plasma and stimulated plasma can lead to distinguishing recent seroconversion from older infections, by the increase in antibody levels found in the stimulated plasma (the Stimulation Index). The increased antibody levels at the early stages of seroconversion stem from the fact that the antibody production in vivo is not at full force, and thus additional activation in vitro leads to higher levels of antibodies in the stimulated-plasma. Later on, the immune activation and antibody production are at such high levels in the body that the levels of antibodies measured in the stimulated-plasma do not differ from those in the regular plasma.

Therefore, the Stimulation Index (SI), defined in one embodiment as the ratio of stimulated to unstimulated antibody levels, measured by a semi-quantitative assay may be used as a novel biomarker for testing for early stages of infection.

There has been, to date, no assays developed for determining/estimating the time of infection (ITT) and/or how early is the stage of infection at the time of first seropositive test result and diagnosis of infection. In defining early infection, there is an adverse trade-off between performance characteristics, and suitably meaningful Mean Duration of Early Infection could suffer from low specificity (or high rate of false early diagnosis). The presented analysis of both the rate of false early diagnosis and Mean Duration of Early Infection, considered together, suggest that an ITT, of utility in diagnosing early infection and determining the time of infection, may be achievable using stimulation methods and/or stimulation devices as described herein or those known in the art. These promising results suggest a fundamentally new assay, using a new type of biomarker for constructing Infection Time Tests (ITT) for diagnosis of early infection and determining time of infection, and support a broadening of the spectrum of biomarkers conventionally used in HIV diagnosis.

### Example 2

### Determination of the Correlation Between SI and Time Since Infection

In a large scale follow up study in a very high risk population, new infections are detected, and are followed for ˜15 months and their SI is recorded at set intervals every week for the first 3-6 months, and monthly thereafter until the SI reaches the “no stimulation” threshold.

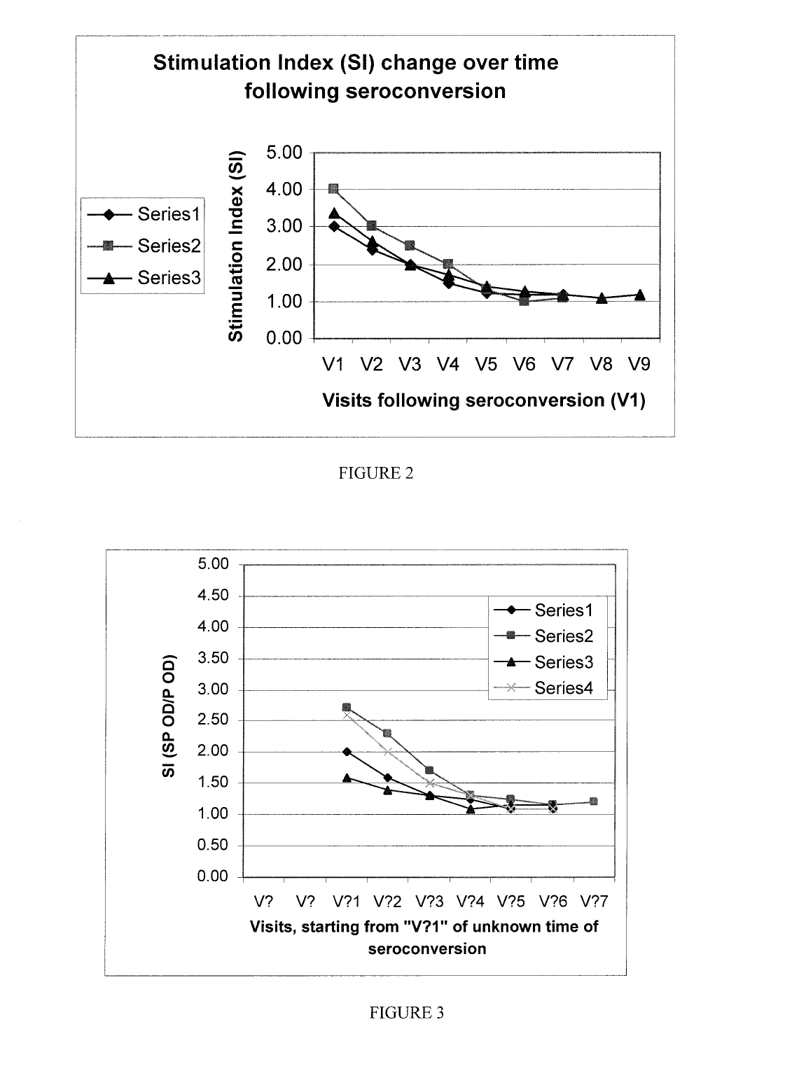

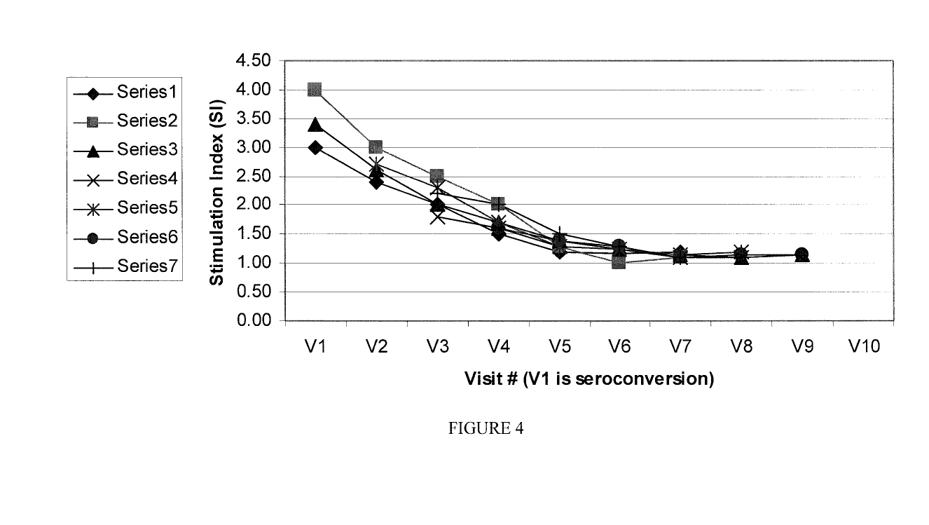

The SI drops rapidly in the first few weeks, and continues to drop for several more months, until it “settles” at a set-SI, which is characteristic of the established infection (FIG. 2). A set of results from a statistically significant number of new infections followed over time (as determined by one skilled in the art) provides tools to determine the types of SI (in this example 1.1, 1.15, or 1.2) and their correlating Mean Duration of Early Infection (in this example 6 or 7 months respectively) to be used for that population (and others, as can be determined by one skilled in the art) to determine the time of infection/seroconversion and/or if the patient is in the early stages of the infection when the time of seroconversion is not known (FIG. 3). In the four examples, the SI is greater than SI for early infection in FIG. 2, and thus they are all early infections. Another example is determining the time of infection by “super imposing” the slope of the SI in the unknown samples over the established slopes of a set of known times of seroconversion (FIG. 4), thus estimating or determining the time of seroconversion. In this example, 2 tested seroconverted ˜1 month earlier, one ˜2 months earlier, and one ˜3 months earlier.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-68. (canceled)

69. A method of determining the time elapsed since an initial microbial infection of a subject comprising the steps of:
a) determining the anti-microbial immunoreactivity in a first aliquot of a blood sample obtained from a subject, wherein a detectable anti-microbial immunoreactivity indicates that said subject is infected;
b) stimulating a second aliquot of said blood sample to produce anti-microbial antibodies in vitro and determining the anti-microbial immunoreactivity in said second aliquot of said blood sample;
c) dividing a value representing the stimulated anti-microbial immunoreactivity obtained in step (b) by a value representing the anti-microbial immunoreactivity obtained in step (a), thereby determining a stimulation index (SI) value; and
d) determining the time elapsed since said initial microbial infection based on the SI value obtained in step (c), wherein there is an inverse correlation between the SI value and the time elapsed since said microbial infection.

70. The method of claim 69, wherein said stimulating step comprises incubating said second aliquot in a media comprising one or more activators of immune cells, in a media comprising one or more activators of microbial-specific cells, or a combination thereof.

71. The method of claim 70, wherein said activator is a mitogen, a microbial derived peptide, lectin, bacterial endotoxin, a virus, lipid A, a cytokine, or a lymphokine.

72. The method of claim 71, wherein said mitogen is phytohaemagglutinin (PHA), concanavalin A (conA), lipopolysaccharide (LPS), pokeweed mitogen (PWM), or a combination thereof.

73. The method of claim 69, wherein said step of determining comprises performing an antibody assay on each aliquot of said blood sample, wherein said antibody assay comprises an enzyme linked immunosorbent assay, an enzyme linked assay, a blot, a luminescence assay, a fluorescence assay or an immunofluorescence assay.

74. The method of claim 69, wherein said microbial infection is an HIV infection.

75. The method of claim 69, further comprising the step of formulating a correlation between SI values and time elapsed since infection prior to step (a) based on a population of subjects for whom both SI value and time elapsed since infection is known.

76. The method of claim 69, further comprising the steps of (a) determining the ratio of in vitro stimulated anti-microbial immunoreactivity and un-stimulated anti-microbial immunoreactivity in a second blood sample from said subject, wherein said ratio is the stimulation index (SI), and (b) calculating the slope of the change in SI calculated for the first and second blood samples, wherein the slope is used to determine the time of said microbial infection.

77. The method of claim 76, wherein said second blood sample is acquired approximately one month after the first blood sample.

78. A method of distinguishing between an early and an established microbial infection in a subject comprising the steps of:
a) determining the anti-microbial immunoreactivity in a first aliquot of a blood sample obtained from a subject, wherein a detectable anti-microbial immunoreactivity indicates that said subject is infected;
b) stimulating a second aliquot of said blood sample to produce anti-microbial antibodies in vitro and determining the anti-microbial immunoreactivity in said second aliquot of said blood sample;
c) dividing a value representing the stimulated anti-microbial immunoreactivity obtained in step (b) by a value representing the anti-microbial immunoreactivity obtained in step (a), thereby determining a stimulation index (SI) value; and
d) determining if the SI value obtained in step (c) is above a pre-determined threshold value, wherein a value below said threshold indicates that the subject has an established infection and a value above said threshold indicates that the subject is in the early stages of the infection,
thereby distinguishing between an early and an established microbial infection in said subject.

79. The method of claim 78, wherein said stimulating step comprises incubating said second aliquot in a media comprising one or more activators of immune cells, in a media comprising one or more activators of microbial-specific cells, or a combination thereof, wherein said stimulating step comprises inducing polyclonal activation of peripheral blood mononuclear cells.

80. The method of claim 79, wherein said activator is a mitogen, a viral-derived peptide, lectin, bacterial endotoxin, a virus, lipid A, a cytokine, a lymphokine, or a combination thereof.

81. The method of claim 80, wherein said mitogen is phytohaemagglutinin (PHA), concanavalin A (conA), lipopolysaccharide (LPS), pokeweed mitogen (PWM), or a combination thereof.

82. The method of claim 78, wherein said step of determining comprises performing an antibody assay on each aliquot of said blood sample, wherein said antibody assay comprises an enzyme linked immunosorbent assay, an enzyme linked assay, a blot, a luminescence assay, a fluorescence assay or an immunofluorescence assay.

83. The method of claim 78, wherein said microbial infection is an HIV infection.

84. The method of claim 78, further comprising the step of formulating a correlation between SI values and time of infection prior to step (a) based on a population of subjects for whom both SI value and time of infection is known.

85. The method of claim 78, further comprising the steps of (a) determining the ratio of in vitro stimulated anti-microbial immunoreactivity and un-stimulated anti-microbial immunoreactivity in a second blood sample from said subject, wherein said ratio is the stimulation index (SI), and (b) calculating the slope of the change in SI calculated for the first and second blood samples, wherein there is an inverse correlation between the value of said slope and the time of said microbial infection.

86. The method of claim 85, wherein said second blood sample is acquired approximately one month after the first blood sample.

87. A kit for determining the time of an initial microbial infection of a subject or for distinguishing between an early and an established microbial infection in a subject comprising: two containers for collecting whole blood samples, wherein the second container comprises a media comprising one or more activators of microbial-specific lymphocytes, non-specific lymphocytes, or a combination thereof, an assay for the detection of one or more products of said lymphocytes, and instructions for use.

88. The kit of claim 87, wherein said assay comprises an enzyme linked immunosorbent assay, an enzyme linked assay, a blot, a luminescence assay, a fluorescence assay or an immunofluorescence assay.

89. The kit of claim 87, wherein said microbial infection is an HIV infection.

90. The kit of claim 87, wherein said instructions for use comprise an algorithm for determining the Stimulation Index (SI) values, an algorithm for determining the time of infection based on the Stimulation Index (SI) value(s), an algorithm for distinguishing between an early and an established microbial infection based on the Stimulation Index (SI) value, or a combination thereof.
